(19) United States
(12) Patent Application Publication
(10) Pub. No.: US 2025/0132025 A1
(43) Pub. Date: Apr. 24, 2025

(54) SYSTEMS AND METHODS OF FACILITATING MANAGEMENT OF A DONOR PROCESS

(71) Applicant: Jaison John Ipe, Little Rock, AR (US)

(72) Inventor: Jaison John Ipe, Little Rock, AR (US)

(21) Appl. No.: 18/921,842

(22) Filed: Oct. 21, 2024

Related U.S. Application Data
(60) Provisional application No. 63/591,694, filed on Oct. 19, 2023.

(51) Int. Cl. G16H 40/20; G16H 10/60; G16H 80/00

(52) U.S. Cl. CPC G16H 40/20; G16H 10/60; G16H 80/00

(57) ABSTRACT

The present disclosure provides a method of facilitating management of a donor process. Further, the method may include receiving, using a communication device, a donor information associated with a donor from a data source device. Further, the method may include retrieving, using a storage device, an eligibility data. Further, the method may include analyzing, using a processing device, the donor information, and the eligibility data. Further, the method may include determining, using the processing device, a donor eligibility data associated with the donor based on the analyzing. Further, the donor eligibility data corresponds to a biological donation performable by a donor. Further, the method may include transmitting, using the communication device, the donor eligibility data to a user device.

The current application claims a priority to the U.S. provisional patent application Ser. No. 63/591,694 filed on Oct. 19, 2024.

### FIELD OF THE INVENTION

Generally, the present disclosure relates to the field of data processing. More specifically, the present disclosure relates to methods, systems, apparatuses, and devices for facilitating management of a donor process.

### BACKGROUND THE INVENTION

The donor eligibility and deferral process refers to the set of guidelines and criteria used to determine whether an individual is eligible to donate blood, plasma, platelets, and other biologics at a given time. Donor deferral is necessary to ensure the safety of both the donor and the recipient of the donated material. The donor deferral process usually varies based on the type of donation. The donor deferral process usually involves an assessment, deferral criteria, and a deferral period. The current manual process raises a variety of challenges such as a lengthy process, the process not being evidence-based, staff making inconsistent deferral decisions, staff training issues, and substandard document control with incorrect old versions circulating at different biologics collection centers.

Therefore, there is a need for improved methods, systems, apparatuses, and devices for facilitating management of a donor process that may overcome one or more of the above-mentioned problems and/or limitations.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

The present disclosure provides a method of facilitating management of a donor process. Further, the method may include receiving, using a communication device, a donor information associated with a donor from a data source device. Further, the method may include retrieving, using a storage device, an eligibility data. Further, the method may include analyzing, using a processing device, the donor information, and the eligibility data. Further, the method may include determining, using the processing device, a donor eligibility data associated with the donor based on the analyzing. Further, the donor eligibility data corresponds to a biological donation performable by a donor. Further, the method may include transmitting, using the communication device, the donor eligibility data to a user device.

The present disclosure provides a system for facilitating management of a donor process. Further, the system may include a communication device. Further, the communication device may be configured to receive a donor information associated with a donor from a data source device. Further, the communication device may be configured to transmit a donor eligibility data to a user device. Further, the system may include a processing device communicatively couple with the communication device. Further, the processing device may be configured to analyze the donor information and an eligibility data. Further, the processing device may be configured to determine the donor eligibility data associated with the donor based on the analyzing. Further, the donor eligibility data corresponds to a biological donation performable by a donor. Further, the system may include a storage device communicatively coupled with the processing device. Further, the storage device may be configured to retrieve the eligibility data.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 illustrates a flowchart of a method 300 of facilitating management of a donor process, in accordance with some embodiments.

FIG. 4 illustrates a flowchart of a method 400 of facilitating management of a donor process including identifying, using the processing device 604, the index data corresponding to the query data, in accordance with some embodiments.

FIG. 5 illustrates a flowchart of a method 500 of facilitating management of a donor process including analyzing, using the processing device 604, the biological sample data, in accordance with some embodiments.

FIG. 6 illustrates a block diagram of a system 600 of facilitating management of a donor process, in accordance with some embodiments.

FIG. 7 illustrates a graphical user interface for facilitating management of a donor process, in accordance with some embodiments.

FIG. 8 illustrates a graphical user interface for facilitating management of a donor process, in accordance with some embodiments.

FIG. 9 is an exemplary method 1000 of facilitating management of a donor process, in accordance with some embodiments.

FIG. 10 is a continuation of FIG. 9.

FIG. 11 is a continuation of FIG. 9.

FIG. 12 is a continuation of FIG. 9.

FIG. 13 illustrates an exemplary illustration 1300 of a plurality of prompts for obtaining additional information for facilitating management of a donor process, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denote “at least one” but do not exclude a plurality unless the contextual use dictates otherwise. When used herein to join a list of items, “or” denotes “at least one of the items” but does not exclude a plurality of items of the list. Finally, when used herein to join a list of items, “and” denotes “all of the items of the list”.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g., a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g., Windows, Mac OS, Unix, Linux, Android, etc.) in order to provide a user interface (e.g., GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g., username, password, passphrase, PIN, secret question, secret answer, etc.) and/or possession of a machine readable secret data (e.g., encryption key, decryption key, bar codes, etc.) and/or possession of one or more embodied characteristics unique to the user (e.g., biometric variables such as, but not limited to, fingerprint, palm-print, voice characteristics, behavioral characteristics, facial features, iris pattern, heart rate variability, evoked potentials, brain waves, and so on) and/or possession of a unique device (e.g., a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g., transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g., the server computer, a client device, etc.) corresponding to the performance of the one or more steps, environmental variables (e.g., temperature, humidity, pressure, wind speed, lighting, sound, etc.) associated with a device corresponding to the performance of the one or more steps, physical state and/or physiological state and/or psychological state of the user, physical state (e.g., motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g., a real-time clock), a location sensor (e.g., a GPS receiver, a GLONASS receiver, an indoor location sensor, etc.), a biometric sensor (e.g., a fingerprint sensor), an environmental variable sensor (e.g., temperature sensor, humidity sensor, pressure sensor, etc.) and a device state sensor (e.g., a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g., initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosed system is an electronic donor eligibility and deferral decision engine that improves donor experience by lowering wait times with quicker decisions made during the biologics donation process. The present disclosed system seeks to provide users with a centralized and standardized enterprise-wide or national repository for donor eligibility and deferral decisions. In order to accomplish this the present disclosed system comprises a user account that is utilized to associate information with a donor condition. Further, the GUI presents a user with an algorithm-based search and decision engine to easily search through a database. Additionally, the online server hosts a database containing various information about donor eligibility and deferral conditions. The online server also hosts the software algorithm that evaluates multiple donor conditions and provides eligibility and deferral decisions. Thus, the present disclosed system is a donor eligibility and deferral decision engine that provides donor center and hospital-based blood bank staff with an online tool to make informed and evidence-based decisions regarding donor medical conditions, donor medications, and other factors including, but not limited to donor medical procedures, donor behaviors, and donor travel.

The present disclosed system is a tool that provides an easy-to-use search engine for medical staff to query a list of over 2000 medical conditions and medications for appropriate donor eligibility and deferral. An objective of the present disclosed system is to provide users with a donor deferral electronic decision engine to improve donor safety by minimizing biologics collection errors. The present disclosed system intends to provide users with an application that further minimizes the amount of time spent by medical directors and staff to make donor eligibility and deferral decisions. To accomplish this the present disclosed system comprises a user account, a GUI, a software-based algorithm and decision engine, and an online server. Many of these components allow for the user to query a database to make appropriate eligibility and deferral decisions. The user account, GUI, and remote server that runs the decision engine algorithm, all remotely communicate to make information available to a user when searching a database through the GUI. Thus, the present disclosed system is a donor eligibility and deferral application that provides donor center and hospital-based biologics collection staff with an electronic tool to make informed and evidence-based decisions regarding donor medical conditions, donor medications, and other factors involving donor behavior and travel.

The present disclosed system begins with a user creating a user account. The user navigates to the donor eligibility and deferral website and creates an account filling in personal information. The user account is approved or denied based on the information provided. In its preferred embodiment, the user profile allows the present disclosed system to make eligibility and deferral information easy to find and allows for the eligibility and deferral information version and change control to be seamless. The user is then prompted when first accessing the donor deferral website application to log into their user account once their user account is approved. It should be further noted that the user account can be created in various ways while still staying within the scope of the present disclosed system.

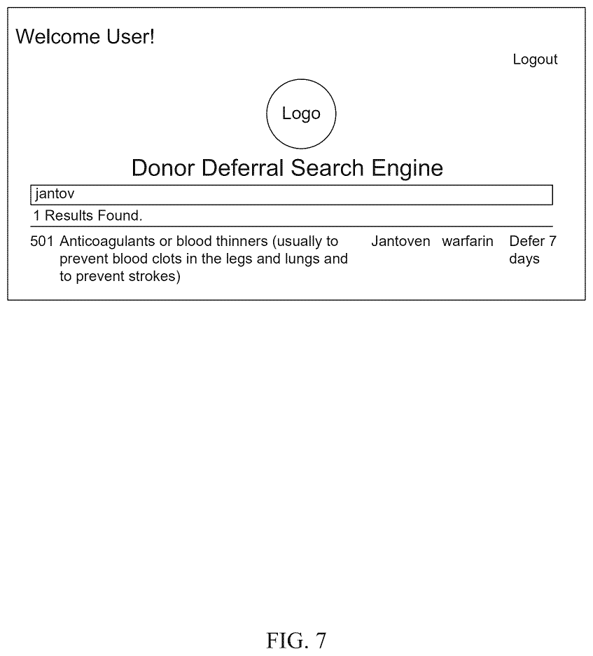

FIG. 7 illustrates a graphical user interface for facilitating management of a donor process, in accordance with some embodiments.

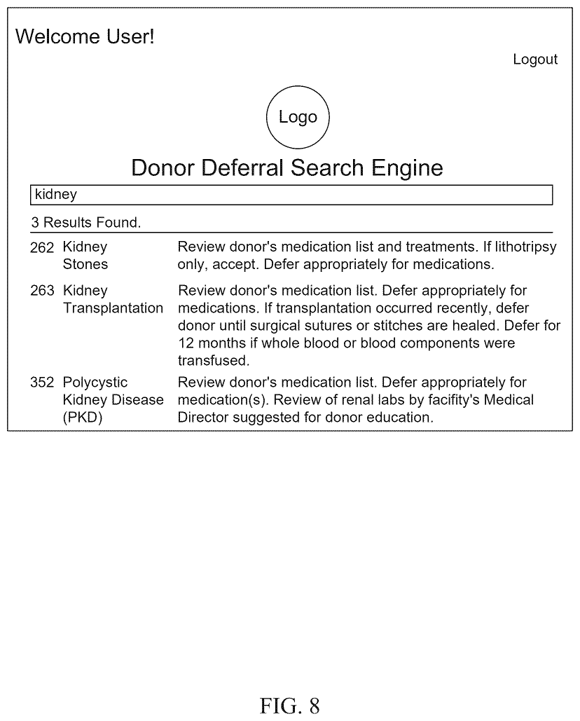

The GUI remotely communicates and provides a login screen to the user for accessing their user account. In its preferred embodiment, the GUI provides the user with a donor eligibility and deferral search engine. The donor deferral search engine allows the user to enter a medical condition or medication into the search bar. Once the term is searched several relevant search results appear with a result reference number. Each result comprises a description of the medication or medical condition, a medication brand name, a medication generic name, and a deferral recommendation. For a medical condition after the term is searched within the search bar all results with the term in the table are displayed, result reference numbers are displayed along with a description or medical condition name, and a deferral recommendation (as shown in FIG. 8). This design allows for lowering delays for decisions on donors without a disparate process that allows for everyone across an organization to utilize one single application and receiving a single eligibility and deferral recommendation.

FIG. 8 illustrates a graphical user interface for facilitating management of a donor process, in accordance with some embodiments.

The online server hosts the backend database and the algorithm-based decision engine that has the data and information about each deferral condition. The database stores the information about each deferral condition with appropriate formatting and their associated donor deferral decisions. The online server allows for the present disclosed system to streamline staff training so that the application will easily update for every user as any new version is uploaded or updated. Furthermore, as a new version is raised for new regulations there is a change in the way a new donor is deferred. In its preferred embodiment, the database is proprietary and is utilized to process information to make an informed decision on a donor deferral. As the database is updated the new version is pushed out to every user to ensure the information available is correct and up to date. This allows the deferral process to become standardized and streamlines staff training and version and change control of deferral information. Further, the present disclosed system minimizes error and provides easy access to correct eligibility and deferral information and the decision engine on the online server.

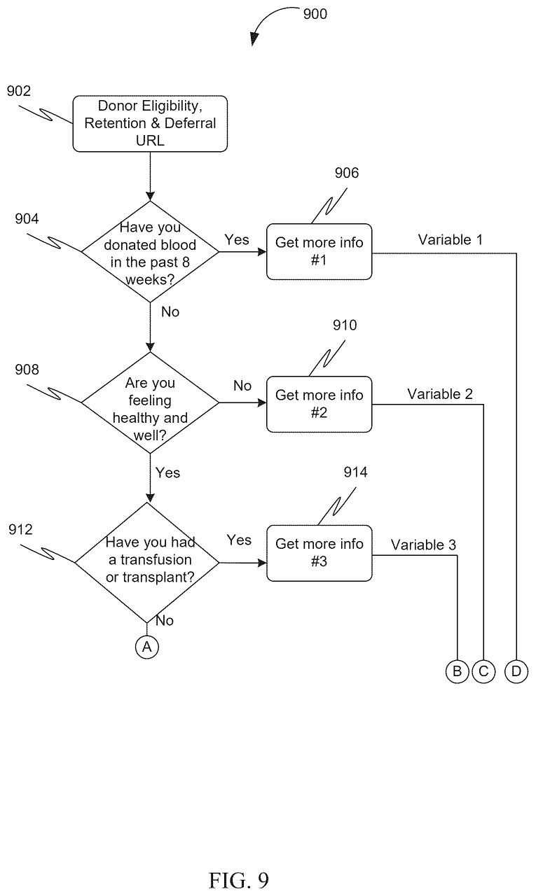

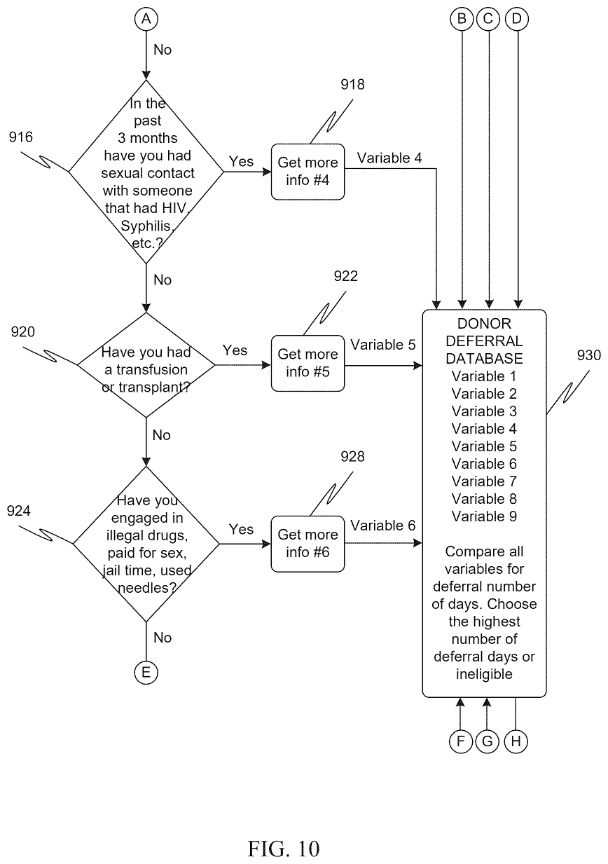

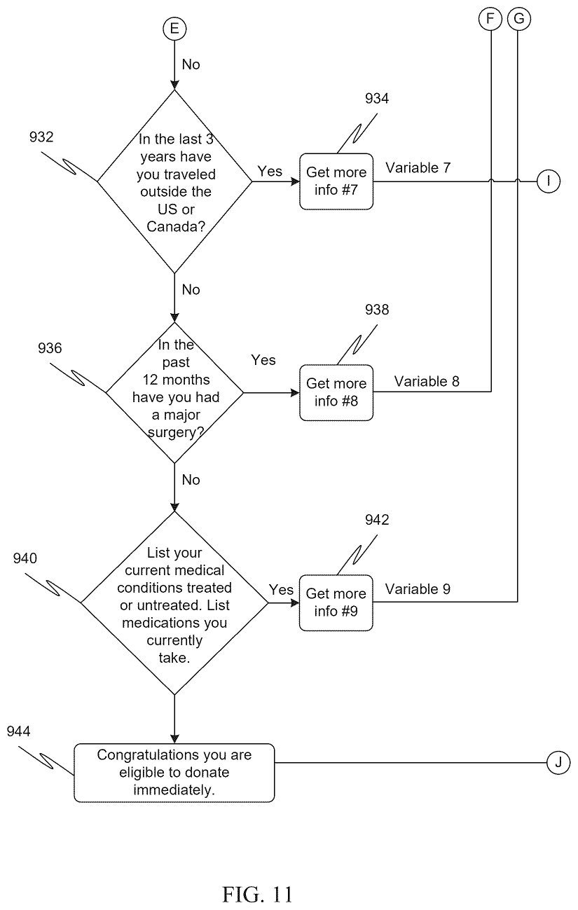

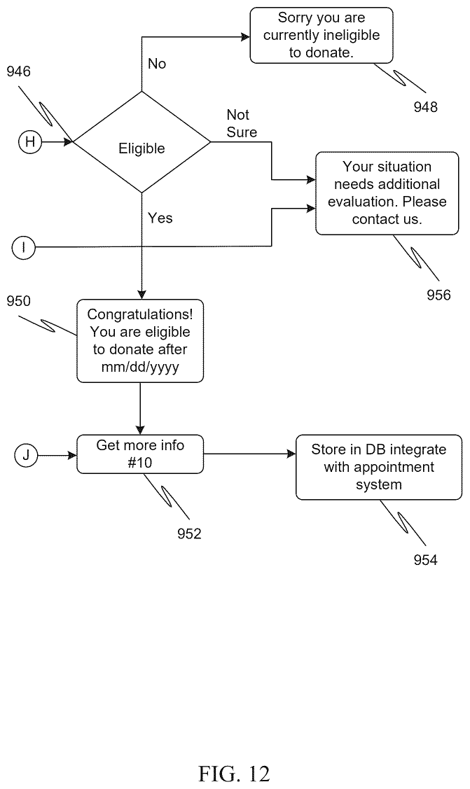

FIGS. 9-12 outline the algorithm used by the software decision engine to evaluate the multiple donor conditions and provide the appropriate eligibility and deferral decision. With all the components working in tandem with each other, the present disclosed system is a donor eligibility and deferral application that provides donor centers and hospital-based biologics collection staff with an online tool to make informed and evidence-based decisions regarding donor behavior, medical conditions and medications used by donors.

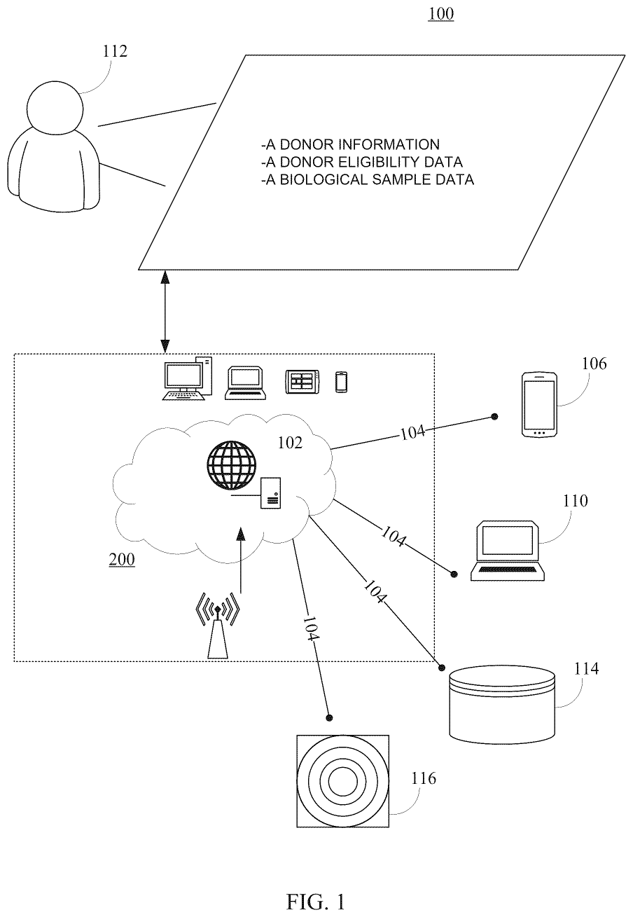

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

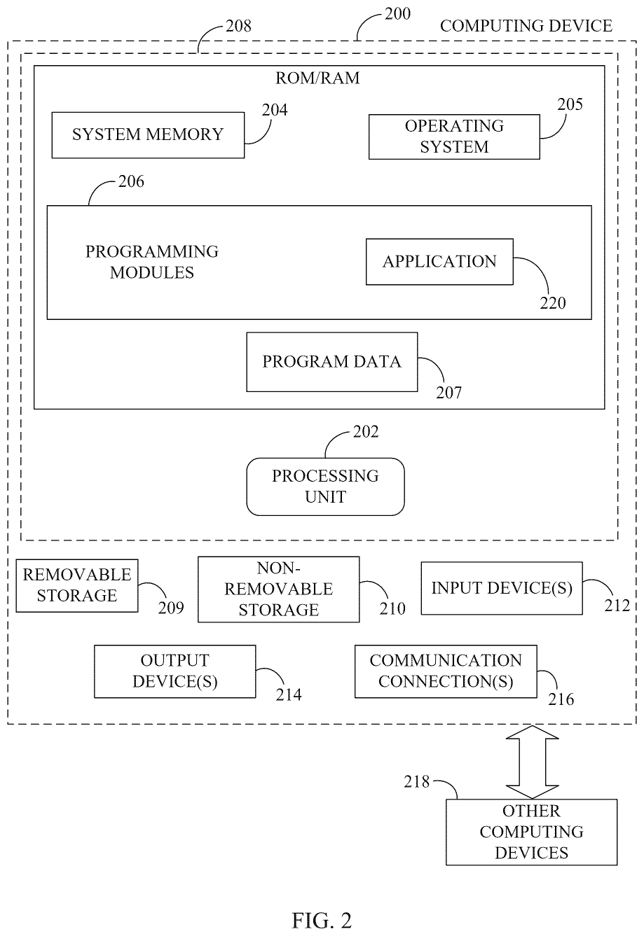

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g., random-access memory (RAM)), non-volatile (e.g., read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable, and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD), or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media.

Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

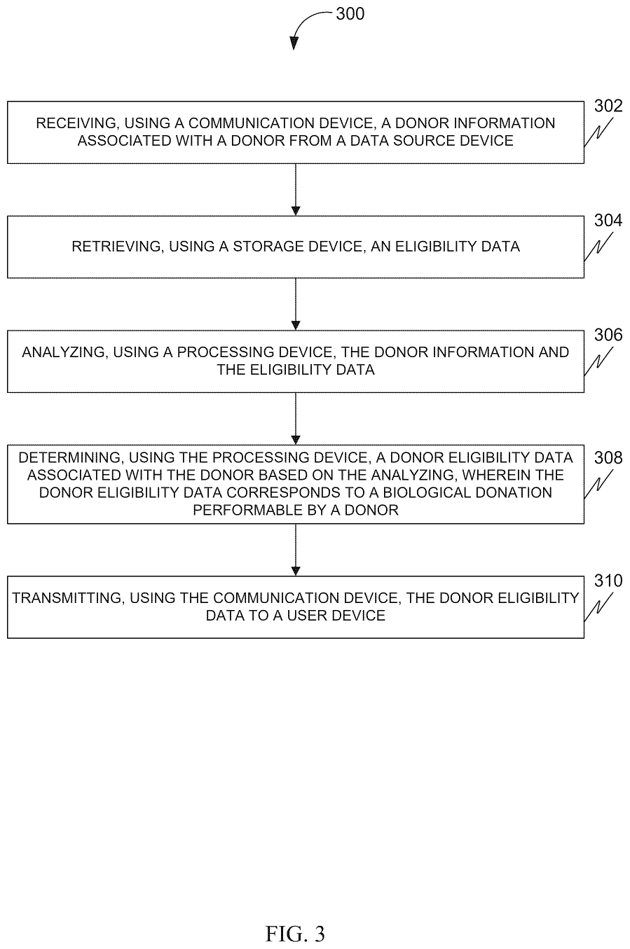

FIG. 3 illustrates a flowchart of a method 300 of facilitating management of a donor process, in accordance with some embodiments.

In some embodiments, the donor process includes one or more of determining a donation eligibility, determining a deferral criteria, and determining a deferral period. In some embodiments, the management of a donor process facilitates minimization of a biologics collection errors.

Accordingly, the method 300 may include a step 302 of receiving, using a communication device 602, a donor information associated with a donor from a data source device 608.

In some embodiments, the communication device 602 may be an electronic device which may be configured to one or more of transmit and receive a data. In some embodiments, the one or more of transmitting and receiving of the data may be wireless. In some embodiments, the donor includes a person intending to perform a biological donation. In some embodiments, the donor information includes one or more of a name, an age data, a blood group data, a medical history data, a medication data, and an additional data. In some embodiments, the donor information includes one or more of a medication data, a medical condition data, and a donor condition data. In some embodiments, the data source device 608 includes a smart device, a computer, and a server. In some embodiments, the data source device 608 may be associated with one or more of a user, a medical staff, a donation center staff, and the donor. In some embodiments, the receiving of the donor information may be based on receiving of a query. Further, the query includes the donor information.

Further, the method 300 may include a step 304 of retrieving, using a storage device 606, an eligibility data.

In some embodiments, the storage device 606 includes a non-volatile memory. In some embodiments, the storage device 606 may be associated with a cloud infrastructure. In some embodiments, the eligibility data includes a data corresponding to more than 2000 medical condition and medication. In some embodiments, the data includes one or more of an eligibility and a deferral. In some embodiments, the eligibility data may be in accordance with a medical association. In some embodiments, the storage device 606 includes a proprietary database.

Further, the method 300 may include a step 306 of analyzing, using a processing device 604, the donor information and the eligibility data.

In some embodiments, the processing device 604 may be an electronic device which may be configured to execute a set of instruction. In some embodiments, the analysis of the donor eligibility data and the eligibility data may be based on a machine learning model.

Further, the method 300 may include a step 308 of determining, using the processing device 604, a donor eligibility data associated with the donor based on the analyzing. Further, the donor eligibility data corresponds to a biological donation performable by a donor.

In some embodiments, the biological donation includes donation of one or more of a blood, a plasma, a platelet, and other biological derived products.

Further, the method 300 may include a step 310 of transmitting, using the communication device 602, the donor eligibility data to a user device 610.

In some embodiments, the user device 610 includes one or more of a smart device, a computer, and a server.

In some embodiments, the donor eligibility data includes an eligible biological product indicator corresponding to an eligible biological product. Further, the eligible biological product includes a biological product eligible to be received from the donor.

In some embodiments, the donor eligibility data includes two or more donor information and two or more deferral periods corresponding to each of the two or more donor information. Further, the method 300 further includes transmitting, using the communication device 602, a user interface to the user device 610. Further, the user interface may be configured to present the two or more donor information and the two or more deferral periods.

In some embodiments, the data source device 608 includes an electronic health records database. Further, the method 300 further includes transmitting, using the communication device 602, a user identifier corresponding to the donor to the electronic health records database.

In some embodiments, the data source device 608 includes a medical instrumentation device which may be configured to generate a medical data corresponding to the donor. Further, the medical data represents a physiological condition of the donor. Further, the medical instrumentation device may be configured for transmitting the medical data. Further, the donor information includes the medical data.

In some embodiments, the medical instrumentation device includes one or more of an oxygen meter, a blood pressure monitor, a temperature sensor, and an EKG monitor.

In some embodiments, the method 300 may further include transmitting, using the communication device 602, a user interface to the user device 610 comprising a user processing device, a user communication device, and a user presentation device. Further, the user presentation device may be configured to present the user interface. Further, the user interface may be configured to present the donor eligibility data in a tabular format.

In some embodiments, the user interface may be further configured to present one or more of the donor eligibility data, the donor information, a medication data, a medical condition, and a deferral data.

In some embodiments, the analyzing of the donor information and the eligibility data may be based on a deferral algorithm.

In some embodiments, the method 300 may further include storing, using a storage device 606, the donor information and the donor eligibility data. Further, the storing may be based on the donor eligibility data.

In some embodiments, the method 300 may further include transmitting, using the communication device 602, two or more prompts. Further, the donor information includes two or more responses to the two or more prompts.

In some embodiments, the two or more prompts includes a donation history prompt, a current health prompt, a medical procedure history prompt, a personal information prompt, a travel history prompt, and a current medication prompt.

In some embodiments, the donor eligibility data includes a deferring data corresponding to a deferral of the biological donation. Further, the deferring data indicates an ineligibility time period associated with the donor. Further, the donor may be not eligible to donate a biological product at least till expiration of the ineligibility time period from a time of an initiation of the donor process.

In some embodiments, the method 300 may further include transmitting, using the communication device 602, a user interface data to the user device 610 comprising a user processing device, a user communication device, and a user presentation device. Further, the user presentation device may be configured to present a user interface based on the user interface data. Further, the user interface may be configured to receive the donor information. Further, the user communication device may be configured to transmit the donor information to the communication device 602. In some embodiments, the user interface may be further configured to present two or more options. Further, the receiving of the donor information may be based on the two or more options.

In some embodiments, the method 300 may further include receiving, using a communication device 602, a login data from the data source device 608. Further, the method 300 further includes verifying, using the processing device 604, a user based on the login data. Further, the receiving of the donor information may be based on the verification of the user.

In some embodiments, the eligibility data may be stored in a hierarchical data structure comprising two or more eligibility data. Further, each of the two or more eligibility data includes an index data which may be configured to allow accessing a corresponding eligibility data within the hierarchical data structure.

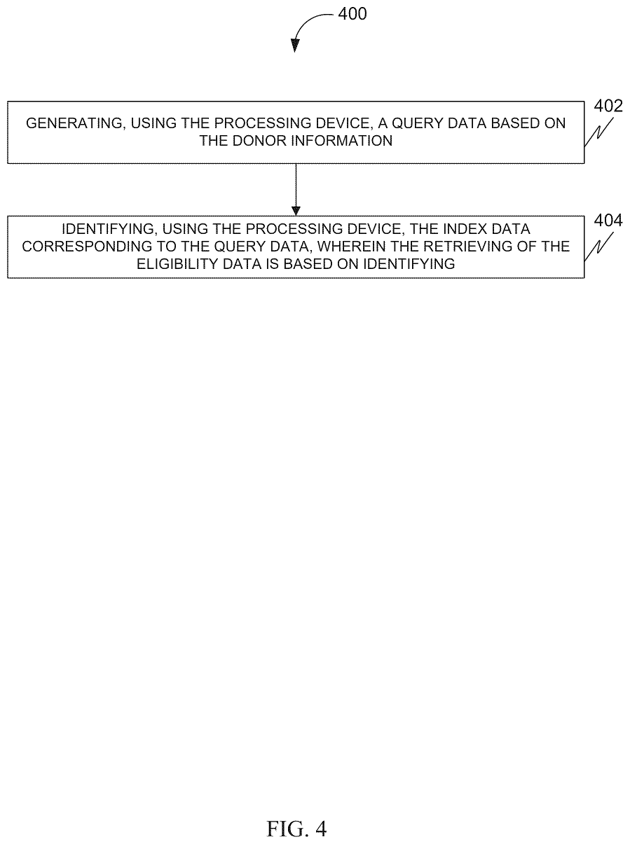

FIG. 4 illustrates a flowchart of a method 400 of facilitating management of a donor process including identifying, using the processing device 604, the index data corresponding to the query data, in accordance with some embodiments.

Further, in some embodiments, the method 400 may include a step 402 of generating, using the processing device 604, a query data based on the donor information. Further, in some embodiments, the method 400 further may include a step 404 of identifying, using the processing device 604, the index data corresponding to the query data. Further, the retrieving of the eligibility data may be based on identifying.

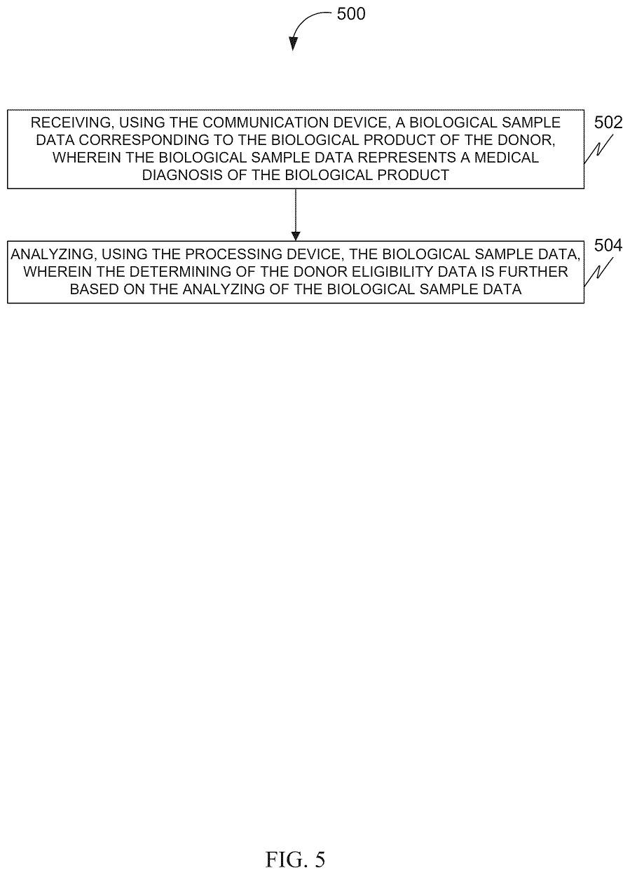

FIG. 5 illustrates a flowchart of a method 500 of facilitating management of a donor process including analyzing, using the processing device 604, the biological sample data, in accordance with some embodiments.

Further, in some embodiments, the method 500 may include a step 502 of receiving, using the communication device 602, a biological sample data corresponding to the biological product of the donor. Further, the biological sample data represents a medical diagnosis of the biological product. Further, in some embodiments, the method 500 may include a step 504 of analyzing, using the processing device 604, the biological sample data. Further, the determining of the donor eligibility data may be further based on the analyzing of the biological sample data.

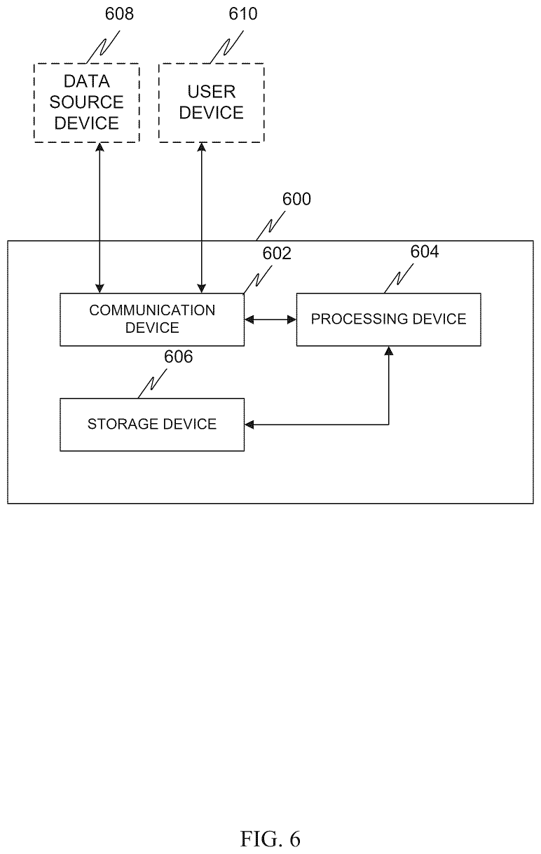

FIG. 6 illustrates a block diagram of a system 600 of facilitating management of a donor process, in accordance with some embodiments.

Accordingly, the system 600 may include a communication device 602. Further, the communication device 602 may be configured to receive a donor information associated with a donor from a data source device 608. Further, the communication device 602 may be configured to transmit a donor eligibility data to a user device 610. Further, the system 600 may include a processing device 604 communicatively coupled with the communication device 602. Further, the processing device 604 may be configured to analyze the donor information and an eligibility data. Further, the processing device 604 may be configured to determine the donor eligibility data associated with the donor based on the analyzing. Further, the donor eligibility data corresponds to a biological donation performable by a donor. Further, the system 600 may include a storage device 606 communicatively coupled with the processing device 604. Further, the storage device 606 may be configured to retrieve the eligibility data.

In some embodiments, the data source device 608 includes an electronic health records database. Further, the communication device 602 may be further configured to transmit a user identifier corresponding to the donor to the electronic health records database.

In some embodiments, the data source device 608 includes a medical instrumentation device which may be configured to generate a medical data corresponding to the donor. Further, the medical data represents a physiological condition of the donor. Further, the medical instrumentation device may be further configured for transmitting the medical data. Further, the donor information includes the medical data.

In some embodiments, the donor eligibility data includes a deferring data corresponding to a deferral of the biological donation. Further, the deferring data indicates an ineligibility time period associated with the donor. Further, the donor may be not eligible to donate a biological product at least till expiration of the ineligibility time period from a time of an initiation of the donor process.

In some embodiments, the communication device 602 may be further configured to transmit a user interface data to the user device 610 comprising a user processing device, a user communication device, and a user presentation device. Further, the user presentation device may be configured to present a user interface based on the user interface data. Further, the user interface may be configured to receive the donor information. Further, the user communication device may be configured to transmit the donor information to the communication device 602.

In some embodiments, the eligibility data may be stored in a hierarchical data structure comprising two or more eligibility data. Further, each of the two or more eligibility data includes an index data which may be configured to allow accessing a corresponding eligibility data within the hierarchical data structure.

Further, in some embodiments, the processing device 604 may be further configured to generate a query data based on the donor information. Further, the processing device 604 may be further configured to identify the index data corresponding to the query data. Further, the retrieving of the eligibility data may be based on identifying.

In some embodiments, the donor eligibility data includes an eligible biological product indicator corresponding to an eligible biological product. Further, the eligible biological product includes a biological product eligible to be received from the donor.

In some embodiments, the donor eligibility data includes two or more donor information and two or more deferral periods corresponding to each of the two or more donor information. Further, the communication device 602 may be further configured to transmit a user interface to the user device 610. Further, the user interface may be configured to present the two or more donor information and the two or more deferral periods.

In some embodiments, the communication device 602 may be further configured to receive a biological sample data corresponding to the biological product of the donor. Further, the biological sample data represents a medical diagnosis of the biological product. Further, the processing device 604 may be further configured to analyze the biological sample data. Further, the determining of the donor eligibility data may be further based on the analyzing of the biological sample data.

FIG. 9-12 is an exemplary method 900 of facilitating management of a donor process, in accordance with some embodiments.

Accordingly, the method 900 may include a step 902 at which a donor accesses the system using Donor Eligibility, Retention & Deferral URL. Subsequently, an evaluation 904 is performed for example by asking a question such as, “Have you donated blood in the past 8 weeks?” if the result of the evaluation 904 is yes, then subsequently, the method 900 includes execution of a step 906 of get more info #1. Further, the information received from step 906 may be stored in a Donor Deferral Database 930 as variable 1. If the result of the evaluation 904 is no, then subsequently, an evaluation 908 is performed for example by asking a question, “Are you feeling healthy and well?” If the result of the evaluation 908 is no, then subsequently the method 900 includes execution of a step 910 of get more info #2. Further, the information received from step 910 may be stored in a Donor Deferral Database 930 as variable 2. If the result of the evaluation 908 is yes, then subsequently, an evaluation 912 is performed for example by asking a question such as, “Have you had a transfusion or transplant?” If the result of the evaluation 912 is yes, then subsequently, the method 900 includes execution of a step 914 of get more info #3. Further, the information received from step 914 may be stored in a Donor Deferral Database 930 as variable 3. If the result of the evaluation 912 is no, then subsequently, an evaluation 916 is performed for example by asking a question such as, “In the past 3 months have you had sexual contact with someone that had HIV, Syphilis, etc.?” If the result of the evaluation 916 is yes, then subsequently, the method 900 includes execution of a step 918 of get more info #4. Further, the information received from step 918 may be stored in a Donor Deferral Database 930 as variable 4. If the result of the evaluation 916 is no, then subsequently, an evaluation 920 is performed for example by asking a question such as, “Have you had a transfusion or transplant?” If the result of the evaluation 920 is yes, then subsequently, the method 900 includes execution of a step 922 of get more info #5. Further, the information received from step 922 may be stored in a Donor Deferral Database 930 as variable 5. If the result of the evaluation 920 is no, then subsequently, an evaluation 924 is performed for example by asking a question such as, “Have you engaged in illegal drugs, paid for sex, jail time, used needles?” If the result of evaluation 924 is yes, then subsequently, the method 900 includes execution of a step 928 of get more info #6. Further, the information received from step 928 may be stored in a Donor Deferral Database 930 as variable 6. If the result of the evaluation 924 is no, then subsequently, an evaluation 932 is performed for example by asking a question such as, “In the last 3 years have you traveled outside the US or Canada?” If the result of the evaluation 932 is yes, then subsequently, the method 900 includes execution of a step 934 of get more info #7. Further, the method 900 includes execution of a step 956 of displaying a message, for example: “Your situation needs additional evaluation. Please contact us.” If the result of the evaluation 932 is no, then subsequently, an evaluation 936 is performed for example by asking a question such as, “In the past 12 months have you had a major surgery?” If the result of the evaluation 932 is yes, then subsequently, the method 900 includes execution of a step 938 of get more info #8. Further, the information received from step 938 may be stored in a Donor Deferral Database 930 as variable 8. If the result of the evaluation 936 is no, then subsequently, an evaluation 940 is performed for example by asking a question such as, “List your current medical conditions treated or untreated. List medications you currently take.” If the result of the evaluation 940 is yes, then subsequently, the method 900 includes execution of a step 942 of get more info #9. Further, the information received from step 942 may be stored in a Donor Deferral Database 930 as variable 9. If the result of evaluation 940 is no, then subsequently, the method 900 includes execution of a step of 944 of displaying a message, for example “Congratulations you are eligible to donate immediately”, Subsequently, the method 900 includes execution of a step of 952 of get more info #10, subsequently method 900 includes execution of the step 954 of Store in DB integrate with appointment system. Further, the method of 900 may include an evaluation 946 based on a plurality of variables storied in Donor Deferral Database 930. If the result of evaluation 946 is no, then subsequently, the method 900 includes execution of a step 948 of displaying a message for example: “Sorry you are currently ineligible to donate”. If the result of evaluation 946 is not sure, then subsequently, the method 900 includes execution of a step 956 of displaying a message for example: “Your situation needs additional evaluation. Please contact us”. If the result of evaluation 946 is yes, then subsequently, the method includes execution of a step 950 of displaying a message for example: “Congratulations!You are eligible to donate after mm/dd/yyyy”, subsequently, the method 900 includes execution of a step 952 of get more info #10, subsequently method 900 includes execution of the step 954 of Store in DB integrate with appointment system.

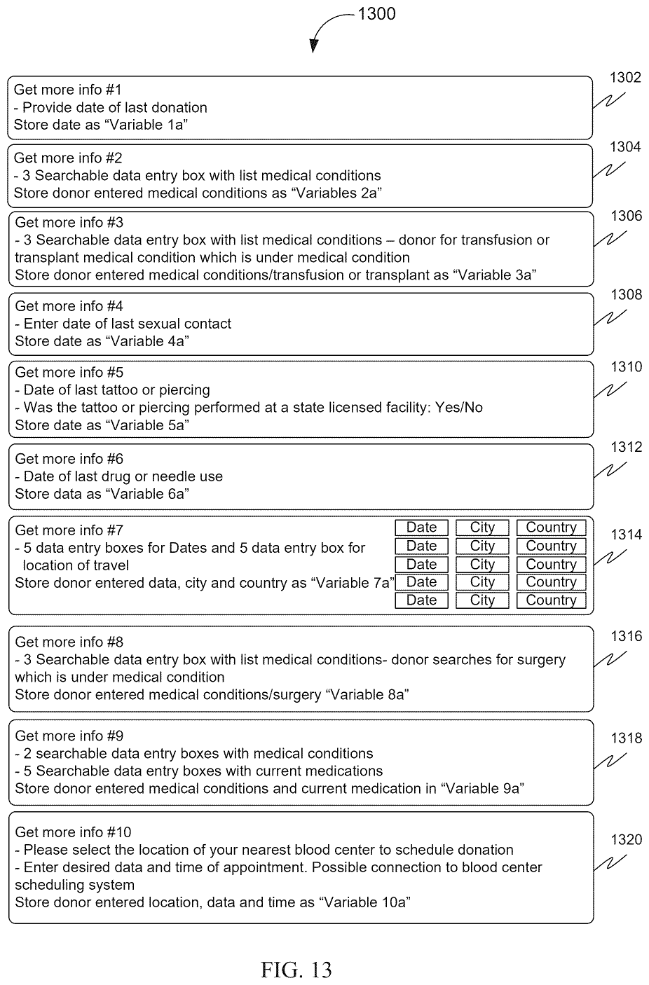

FIG. 13 illustrates an exemplary illustration 1300 of a plurality of prompts for obtaining additional information for facilitating management of a donor process, in accordance with some embodiments.

Accordingly, the exemplary illustration 1300 illustrates an exemplary prompt 1302 corresponding to get more info #1 that prompts the donor to provide date of the last donation. Further, the exemplary method 1300 illustrates an exemplary prompt 1304 corresponding to get more info #2 that prompts the donor to provide a list of medical conditions. Further, the exemplary method 1300 illustrates an exemplary prompt 1306 corresponding to get more info #3 that prompts the donor to provide list of medical conditions associated with transfusion and/or transplant. Further, the exemplary method 1300 illustrates an exemplary prompt 1308 corresponding to get more info #4 that prompts the donor to provide last date of sexual contact. Further, the exemplary method 1300 illustrates an exemplary prompt 1310 corresponding to get more info #5 that prompts the donor to provide date of the last tattoo and/or piercing and whether the tattoo or piercing was performed at a state-licensed facility. Further, the exemplary method 1300 illustrates an exemplary prompt 1312 corresponding to get more info #6 that prompts the donor to provide date of the last drug and/or needle use. Further, the exemplary method 1300 illustrates an exemplary prompt 1314 corresponding to get more info #7 that prompts the donor to provide data associated with the location of travel and date of travel. Further, the exemplary method 1300 illustrates an exemplary prompt 1316 corresponding to get more info #8 that prompts the donor to provide data associated with the surgery of the donor. Further, the exemplary method 1300 illustrates an exemplary prompt 1318 corresponding to get more info #9 that prompts the donor to provide data associated with medical conditions and current medications. Further, the exemplary method 1300 illustrates an exemplary prompt 1320 corresponding to get more info #10 that prompts the donor to provide the preferred location of the donation center and data associated with the preferred appointment date and time.

Although the invention has been explained in relation to its preferred embodiment, it is to be understood that many other possible modifications and variations can be made without departing from the spirit and scope of the invention as hereinafter claimed.

What is claimed is:

1. A method of facilitating management of a donor process, the method comprising:
receiving, using a communication device, a donor information associated with a donor from a data source device;
retrieving, using a storage device, an eligibility data;
analyzing, using a processing device, the donor information and the eligibility data;
determining, using the processing device, a donor eligibility data associated with the donor based on the analyzing, wherein the donor eligibility data corresponds to a biological donation performable by a donor; and
transmitting, using the communication device, the donor eligibility data to a user device.

2. The method of claim 1, wherein the data source device comprises an electronic health records database, wherein the method further comprises transmitting, using the communication device, a user identifier corresponding to the donor to the electronic health records database.

3. The method of claim 1, wherein the data source device comprises a medical instrumentation device configured to generate a medical data corresponding to the donor, wherein the medical data represents a physiological condition of the donor, wherein the medical instrumentation device is further configured for transmitting the medical data, wherein the donor information comprises the medical data.

4. The method of claim 1, wherein the donor eligibility data comprises a deferring data corresponding to a deferral of the biological donation, wherein the deferring data indicates an ineligibility time period associated with the donor, wherein the donor is not eligible to donate a biological product at least till expiration of the ineligibility time period from a time of an initiation of the donor process.

5. The method of claim 1 further comprising transmitting, using the communication device, a user interface data to the user device comprising a user processing device, a user communication device, and a user presentation device, wherein the user presentation device is configured to present a user interface based on the user interface data, wherein the user interface is configured to receive the donor information, wherein the user communication device is configured to transmit the donor information to the communication device.

6. The method of claim 1, wherein the eligibility data is stored in a hierarchical data structure comprising a plurality of eligibility data, wherein each of the plurality of eligibility data comprises an index data configured to allow accessing a corresponding eligibility data within the hierarchical data structure.

7. The method of claim 6 further comprising:
generating, using the processing device, a query data based on the donor information; and
identifying, using the processing device, the index data corresponding to the query data, wherein the retrieving of the eligibility data is based on identifying.

8. The method of claim 1, wherein the donor eligibility data comprises an eligible biological product indicator corresponding to an eligible biological product, wherein the eligible biological product comprises a biological product eligible to be received from the donor.

9. The method of claim 1, wherein the donor eligibility data comprises a plurality of donor information and a plurality of deferral periods corresponding to each of the plurality of donor information, wherein the method further comprises transmitting, using the communication device, a user interface to the user device, wherein the user interface is configured to present the plurality of donor information and the plurality of deferral periods.

10. The method of claim 1 further comprising:
receiving, using the communication device, a biological sample data corresponding to the biological product of the donor, wherein the biological sample data represents a medical diagnosis of the biological product;
analyzing, using the processing device, the biological sample data, wherein the determining of the donor eligibility data is further based on the analyzing of the biological sample data.

11. A system for facilitating management of a donor process, the system comprising:
a communication device configured to:
receive a donor information associated with a donor from a data source device;
transmit a donor eligibility data to a user device;
a processing device communicatively coupled with the communication device, wherein the processing device is configured to:
analyze the donor information and an eligibility data; and
determine the donor eligibility data associated with the donor based on the analyzing, wherein the donor eligibility data corresponds to a biological donation performable by a donor; and
a storage device communicatively coupled with the processing device, wherein the storage device is configured to retrieve the eligibility data.

12. The system of claim 11, wherein the data source device comprises an electronic health records database, wherein the communication device is further configured to transmit a user identifier corresponding to the donor to the electronic health records database.

13. The system of claim 11, wherein the data source device comprises a medical instrumentation device configured to generate a medical data corresponding to the donor, wherein the medical data represents a physiological condition of the donor, wherein the medical instrumentation device is further configured for transmitting the medical data, wherein the donor information comprises the medical data.

14. The system of claim 11, wherein the donor eligibility data comprises a deferring data corresponding to a deferral of the biological donation, wherein the deferring data indicates an ineligibility time period associated with the donor, wherein the donor is not eligible to donate a biological product at least till expiration of the ineligibility time period from a time of an initiation of the donor process.

15. The system of claim 11, wherein the communication device is further configured to transmit a user interface data to the user device comprising a user processing device, a user communication device, and a user presentation device, wherein the user presentation device is configured to present a user interface based on the user interface data, wherein the user interface is configured to receive the donor information, wherein the user communication device is configured to transmit the donor information to the communication device.

16. The system of claim 11, wherein the eligibility data is stored in a hierarchical data structure comprising a plurality of eligibility data, wherein each of the plurality of eligibility data comprises an index data configured to allow accessing a corresponding eligibility data within the hierarchical data structure.

17. The system of claim 16, wherein the processing device is further configured to:
generate a query data based on the donor information; and
identify the index data corresponding to the query data, wherein the retrieving of the eligibility data is based on identifying.

18. The system of claim 11, wherein the donor eligibility data comprises an eligible biological product indicator corresponding to an eligible biological product, wherein the eligible biological product comprises a biological product eligible to be received from the donor.

19. The system of claim 11, wherein the donor eligibility data comprises a plurality of donor information and a plurality of deferral periods corresponding to each of the plurality of donor information, wherein the communication device is further configured to transmit a user interface to the user device, wherein the user interface is configured to present the plurality of donor information and the plurality of deferral periods.

20. The system of claim 11, wherein the communication device is further configured to receive a biological sample data corresponding to the biological product of the donor, wherein the biological sample data represents a medical diagnosis of the biological product, wherein the processing device is further configured to analyze the biological sample data, wherein the determining of the donor eligibility data is further based on the analyzing of the biological sample data.
